(19) United States
(12) Patent Application Publication
(10) Pub. No.: US 2023/0173116 A1
(43) Pub. Date: Jun. 8, 2023

(54) Disinfection Device, Vehicle, And Construction Machine

(71) Applicant: JDC Corporation, Minato-Ku Tokyo (JP)

(72) Inventors: Masakazu Sekiguchi, Tokyo (JP); Hiroshi Obata, Tokyo (JP); Hidetoshi Morimoto, Tokyo (JP); Tsukasa Baba, Tokyo (JP)

(21) Appl. No.: 17/911,623

(22) PCT Filed: Dec. 15, 2020
(86) PCT No.: PCT/JP2020/046793

§ 371 Date: Sep. 14, 2022

Related U.S. Application Data
(60) Provisional application No. 63/040,667, filed on Jun. 18, 2020.

(51) Int. Cl. A61L 2/10; A61L 2/18

(52) U.S. Cl. CPC A61L 2/10; A61L 2/18; A61L 2202/16; A61L 2202/14

(57) ABSTRACT

A disinfection device, a vehicle, and a construction machine capable of preventing infection of infectious diseases are described. The disinfection device includes a fluid supply device that supplies a fluid to a disinfection area and an irradiation device that irradiates the disinfection area with invisible light.

### TECHNICAL FIELD

The present invention relates to a disinfection device, a vehicle, and a construction machine capable of disinfecting, for example, a virus.

### BACKGROUND

Patent Publication No. JP 2018-117708 A discloses a cough detection device that is provided with a motion detection unit and a sound detection unit corresponding to a seat in a living space of an office or a classroom or in a moving body such as a bus, a train, or an airplane, and detects at which seat a person has coughed. Further, Patent Publication No. JP 2018-117708 A discloses that an infectious disease is detected on the basis of a detection result of the cough detection device.

### SUMMARY

However, in Patent Publication No. JP 2018-117708 A, disclosure is limited to the detection of an infectious disease, and measures such as prevention of infection of the infectious disease is not disclosed.

Therefore, an object of the present invention is to provide a disinfection device, a vehicle, and a construction machine capable of preventing infection of infectious diseases.

A disinfection device according to an embodiment of the present invention includes a fluid supply device that supplies a fluid to a disinfection area, and an irradiation device that irradiates the disinfection area with invisible light.

A vehicle according to an embodiment of the present invention includes a detection device that detects a cough or a sneeze of an operator in a cockpit, and a control device that drives at least one of an irradiation device that emits invisible light or a fluid supply device that supplies a disinfectant fluid when the detection device detects a cough or a sneeze.

A construction machine according to an embodiment of the present invention includes a disinfection device that performs at least one of supply of a disinfectant fluid or irradiation with invisible light in a maintenance area where maintenance is performed.

According to a disinfection device of the present invention, the disinfection area can be efficiently disinfected.

A vehicle of the invention can disinfect the vehicle in response to detecting a cough or a sneeze.

A construction machine of the present invention can efficiently disinfect the maintenance area.

### BRIEF DESCRIPTION OF THE DRAWINGS

FIG. 1 is a schematic view illustrating a towed scraper according to a first embodiment of the teachings herein.

FIG. 2 is a schematic view illustrating a cockpit of a towing vehicle according to the first embodiment.

FIG. 3 is a block diagram of a main part of the first embodiment.

FIG. 4 is a diagram illustrating a flowchart executed by a control device of the first embodiment.

FIG. 5 is a schematic view illustrating a rotary crushing device according to the second embodiment.

FIG. 6 is a block diagram of a main part of a second embodiment of the teachings herein.

FIG. 7 is a diagram illustrating a flowchart executed by a control device of the second embodiment.

### DETAILED DESCRIPTION

Hereinafter, a first embodiment of the present invention will be described in detail with reference to the accompanying drawings. Note that the present invention is not limited by the embodiments to be described below.

### First Embodiment

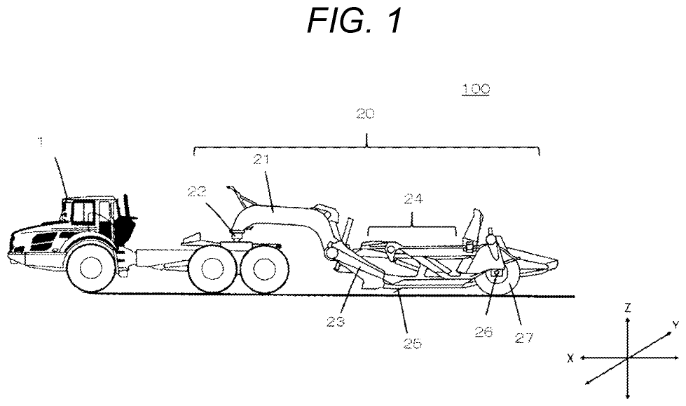

FIG. 1 is a schematic view illustrating a towed scraper 100 according to the present embodiment. The towed scraper 100 of the present embodiment includes a towing vehicle 1 which is a driving vehicle and a scraper vehicle 20. As illustrated in FIG. 1, the towing vehicle 1 tows the scraper vehicle 20, and is connected to the scraper vehicle 20 by a hitch 21 that is a connection device. The hitch 21 is attachable to and detachable from the towing vehicle 1. The hitch 21 includes a flexible ball joint 22 provided at one end on the towing vehicle 1 side and a flexible ball joint (not illustrated) provided at the other end on the scraper vehicle 20 side.

Towing Vehicle

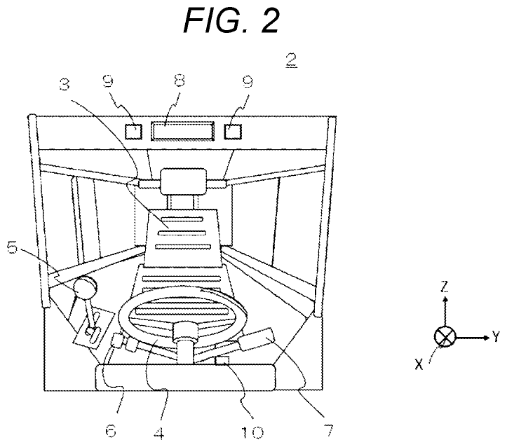

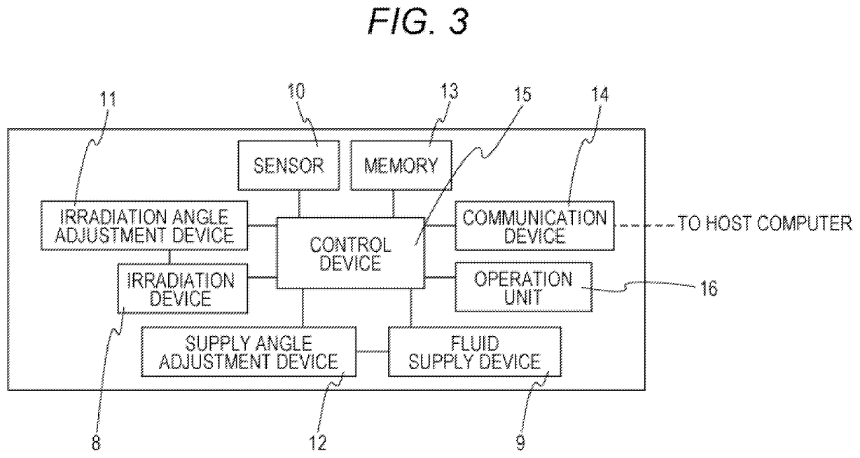

FIG. 2 is a schematic view illustrating a cockpit 2 of the towing vehicle 1. The cockpit 2 in FIG. 2 is a schematic view of the cockpit 2 as viewed from a front side of the towing vehicle 1. FIG. 3 is a block diagram of a main part of the cockpit 2 of the present embodiment. Hereinafter, the cockpit 2 will be described with reference to FIGS. 2 and 3.

In the present embodiment, the cockpit 2 includes a driver's seat 3, a steering wheel 4, a gear shift lever 5, a blinker 6, a wiper 7, an accelerator (not illustrated), a brake (not illustrated), and the like. The cockpit 2 further includes an irradiation device 8 that irradiates an inside of the cockpit 2 with invisible light, a fluid supply device 9 that supplies a fluid into the cockpit 2, and a sensor 10. Note that the layout of each element of the driver's seat 3 is merely an example, and various changes such as changing left and right layouts can be made.

As the towing vehicle 1 of the first embodiment, a dump truck can be adopted, and for example, an articulated dump truck. In such an articulated dump truck, the steering wheel 4, the gear shift lever 5, the blinker 6, the wiper 7, the accelerator (not illustrated), and the brake (not illustrated) have known configurations. Thus, description of the configurations is omitted. In addition, the steering wheel 4, the gear shift lever 5, the blinker 6, the wiper 7, the accelerator (not illustrated), and the brake (not illustrated) may be collectively referred to as an operation unit 16 (see FIG. 3).

The irradiation device 8 is provided in an upper part of the cockpit 2 (for example, a ceiling or a vicinity of the ceiling) and irradiates the cockpit 2 with invisible light such as ultraviolet light from a depth side in the drawing (depth side in an X direction). In the present embodiment, far ultraviolet C waves having 205 to 230 nm are emitted as the ultraviolet light. This is because it is considered that the far ultraviolet C waves having a wavelength of 205 to 230 nm do not reach human cells. Thus, the waves do not harm the human body, but they penetrate bacteria and viruses in the air and on a surface of an object and can disinfect the air and the surface of the object. By adopting the far ultraviolet C waves having the wavelength of 205 to 230 nm, the invisible light can be emitted from the irradiation device 8 even in a case where a driver is in the driver's seat 3.

Note that it is also possible to use the ultraviolet light in a wavelength region other than the wavelength of 205 to 230 nm by controlling the invisible light to be emitted from the irradiation device 8 in a case where there is no driver in the driver's seat 3. Also in this case, the ultraviolet light that can penetrate bacteria and viruses and can perform disinfection is used.

The irradiation device 8 irradiates an area with which the driver comes in contact with or the cockpit 2 (hereinafter collectively referred to as a disinfection area) and irradiates the driver's seat 3, the steering wheel 4, the gear shift lever 5, the blinker 6, the wiper 7, the accelerator (not illustrated), the brake (not illustrated), a door knob (not illustrated), a floor, and the like with the far ultraviolet C waves. To irradiate such a disinfection area with the far ultraviolet C waves, a plurality of irradiation devices 8 may be provided or the irradiation device 8 may be provided on the floor side to emit the far ultraviolet C waves from the floor side toward the ceiling side. Further, an irradiation angle adjustment device 11 (see FIG. 3) may be provided to adjust an irradiation angle of the irradiation device 8 in a Y direction or a Z direction. Note that it is also possible to neutralize bacteria and viruses floating in the cockpit 2 by the emission of the far ultraviolet C waves by the irradiation device 8.

An example of the fluid supply device 9 is a gas supply device that supplies a compressed gas (for example, air) to the disinfection area. A foreign substance such as soil may adhere to a hand or a shoe of the driver who drives the towed scraper 100, and a foreign substance such as soil may adhere to the disinfection area. For example, the bacteria and viruses may directly adhere to the steering wheel 4, may adhere to soil attached to the steering wheel 4, or may adhere to both the steering wheel and the soil. The irradiation device 8 can neutralize the bacteria and viruses attached to a surface of the soil, but it may not be able to neutralize the bacteria and viruses existing between the steering wheel 4 and the soil in some cases.

Therefore, in the first embodiment, after the irradiation device 8 neutralizes the bacteria and viruses attached to the surface of the soil, the fluid supply device 9 blows off the disinfected soil with the compressed gas. The irradiation device 8 can neutralize the bacteria and viruses directly attached to the operation unit 16 such as the steering wheel 4 by irradiating the disinfection area from which the soil has been blown off with the far ultraviolet C waves.

The fluid supply device 9 may supply a disinfectant liquid to the disinfection area in place of or in combination with the compressed gas. As the disinfectant liquid, various kinds of liquids such as an alcohol-based solution, an aldehyde-based solution, a chlorine-based solution, an iodine-based solution, an oxidant-based solution, and a quaternary ammonium salt-based solution can be used. In the present embodiment, an alcohol-based disinfectant solution is used in the case where the driver is in the driver's seat 3, and a non-alcohol-based disinfectant solution is used in the case where the driver is not in the driver's seat 3. Thus, the human body of the driver is not damaged. In this case, a plurality of containers may be provided in the fluid supply device 9, the alcohol-based disinfectant solution may be placed in one container, a non-alcohol-based disinfectant solution may be placed in the other container, and the disinfectant solution in the one container may be selected from the plurality of containers depending on whether the driver is present. Note that the number of containers may be three or more, and two or more kinds of disinfectant solutions may be supplied.

Further, even in a case of using the disinfectant solution that is not harmful to the human body of the driver, the fluid supply device 9 favorably supplies the disinfectant solution such that the disinfectant solution is not applied to the driver, and particularly favorably supplies the disinfectant solution such that the disinfectant solution is not applied to the face of the driver. Note that the fluid supply device 9 desirably sprays the disinfectant solution in an atomized form by a spray nozzle.

The disinfection area is disinfected by the fluid supply device 9 to efficiently disinfect the disinfection area and to disinfect the disinfection area where the far ultraviolet C waves are blocked by the steering wheel 4 and the like such that the blocked area is not irradiated with the far ultraviolet C waves.

Note that the irradiation angle adjustment device 11 may adjust the irradiation angle of the irradiation device 8 so as not to block the far ultraviolet C waves. Further, a supply angle adjustment device 12 (see FIG. 3) may be provided to adjust a supply angle of the fluid supplied by the fluid supply device 9 in the Y direction or the Z direction.

FIG. 2 illustrates two fluid supply devices 9. One may be a gas supply device that supplies the compressed gas and the other may be a liquid supply device that supplies the disinfectant solution.

Further, both fluid supply devices 9 may be gas supply devices or both fluid supply devices 9 may be liquid supply devices. Further, the fluid supply devices 9 may supply both the compressed gas and the disinfectant solution. Note that the number of fluid supply devices 9 may be one, or three or more, and the fluid supply devices 9 may be disposed on the floor. Further, the irradiation device 8 and the fluid supply devices 9 may be unitized to form one device.

The sensor 10 is a sound detection unit such as a microphone as described in JP 2018-117708 A. The sensor 10 detects a cough or a sneeze of the driver. Whether a sound signal detected by the sensor 10 is a cough or a sneeze can be detected by a frequency analysis for the sound signal on the basis of an output of the sensor 10. In this case, the sound signal detected by the sensor 10 may be wirelessly transmitted to a host computer, and the frequency analysis may be performed by the host computer, or the frequency analysis may be performed by a control device 15 (see FIG. 3).

As the sensor 10, a motion detection unit having an acceleration sensor as described in JP 2018-117708 A may be provided to detect the cough or sneeze of the driver. In this case, an acceleration sensor is favorably provided in the driver's seat 3. A numerical value or a pattern of acceleration when a person cough or sneezes is stored as a reference in a memory of a host computer or a memory 13 (see FIG. 3) on the towing vehicle 1 side, and whether the driver has coughed or sneezed can be detected by comparison with the acceleration detected by the acceleration sensor.

By providing both the microphone and the acceleration sensor as the sensor 10, it is possible to accurately detect the cough or sneeze of the driver. Note that the number of microphones and the number of acceleration sensors may be one or more. While an arrangement place thereof can also be arbitrarily set, the microphone is favorably provided on a front side of the driver (a side facing the face of the driver).

Further, to reduce the cost of the towing vehicle 1, either the microphone or the acceleration sensor may be provided as the sensor 10.

Further, a thermometer may be installed as the sensor 10. In this case, the sensor 10 may descend from a ceiling at the time when the driver sits on the driver's seat so the sensor 10 faces the driver's forehead. As the thermometer, a non-contact type thermometer is favorable. For example, a thermometer using infrared rays can be used but the thermometer is not limited thereto. Note that, to avoid soil from adhering to the sensor 10, the sensor 10 may be covered with a cover at times other than when performing detection by the sensor 10, and the cover may be removed at the time of performing detection by the sensor 10.

When at least one of the irradiation device 8 or the fluid supply device 9 is driven at the timing when the sensor 10 detects the cough or sneeze of the driver, the bacteria or viruses can be immediately neutralized even in a case where the bacteria or viruses are included in the cough or sneeze. Further, when at least one of the irradiation device 8 or the fluid supply device 9 is driven in a case where the sensor 10 detects heat generation of the driver, the bacteria or viruses included in exhalation of the driver can be immediately neutralized.

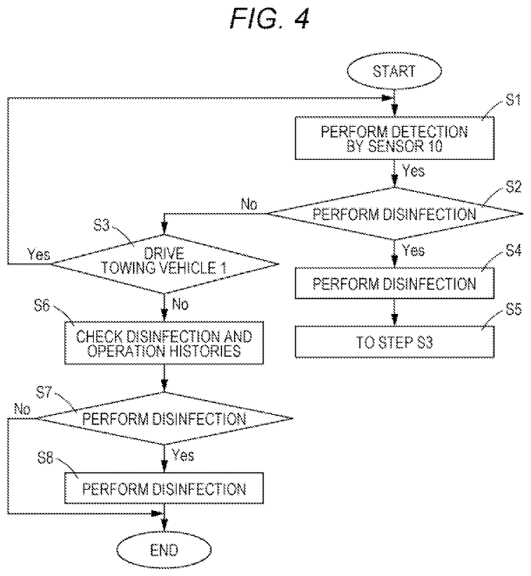

The memory 13 is a nonvolatile memory (for example, a flash memory), and stores the above-described numerical value and pattern of the acceleration, s disinfection history of the cockpit 2, and various data for driving the towing vehicle 1. In addition, the memory 13 stores a program for driving the towing vehicle 1 and various programs such as that implementing a flowchart of FIG. 4 to be described below.

A communication device 14 is a wireless communication unit that accesses a wide area network such as a host computer or the Internet, and in the present embodiment, transmits a detection result of the sensor 10, an irradiation history of the irradiation device 8, a supply history of the fluid supply device 9, an operation history of the operation unit 16, and the like to the host computer. Note that the detection result of the sensor 10, the irradiation history of the irradiation device 8, the supply history of the fluid supply device 9, the operation history of the operation unit 16, and the like may be stored in the memory 13.

The control device 15 includes a central processing unit (CPU) and performs control for neutralizing the bacteria and viruses in the cockpit 2 in addition to control of the towing vehicle 1 and the scraper vehicle 20. Note that the control for neutralizing the bacteria and viruses in the cockpit 2 by the control device 15 will be described below.

Scraper Vehicle

Returning to FIG. 1, the scraper vehicle 20 includes a frame 23, a bowl 24, a scraper 25, an axle 26, wheels 27, and the like, in addition to the above-described hitch 21 and ball joint 22.

The frame 23 is a metal frame that supports a structure such as the bowl 24, and the bowl 24 has an open upper surface and houses an excavated object such as earth and sand excavated by the scraper 25.

The scraper 25 is a blade-shaped or spatula-shaped member for scraping the earth and sand on a traveling surface such as a ground surface, and the scraper 25 is integrally provided with the bowl 24 at a bottom of the bowl 24 in the present embodiment.

Because the bowl 24 and the scraper 25 are integrally provided, the scraper 25 can cut into the ground and excavate the earth and sand by inclining the bowl 24 toward the ground by a hydraulic cylinder (not illustrated). Further, the bowl 24 is provided with the opening (not illustrated), and the excavated object excavated by the scraper 25 is accommodated in the bowl 24 through the opening (not illustrated) when the bowl 24 is inclined toward the ground.

When the excavation by the scraper 25 is completed, the bowl 24 is inclined upward toward the ground by the hydraulic cylinder (not illustrated). By inclining the bowl 24, the scraper 25 is separated from the ground.

The axle 26 rotates by a traction force of the towing vehicle 1. The wheels 27 are connected to both ends of the axle 26 and are a pair of driven wheels that rotate with the rotation of the axle 26. Note that the wheels 27 may be provided in front of and behind the scraper vehicle 20 as front wheels and rear wheels.

Description of Flowchart

The control for neutralizing the bacteria and viruses in the cockpit 2 by the control device 15 of the present embodiment configured as described above will be described with reference to the flowchart of FIG. 4. Note that the process shown by the present flowchart is assumed to be performed when an engine of the towing vehicle 1 is driven.

The control device 15 performs the detection by the sensor 10 to detect whether the driver has coughed or sneezed (step S1). Note that, in step S1, the control device 15 may detect a body temperature of the driver. Note that because there is a case where a plurality of drivers alternately drives the towing vehicle 1, a face recognition device may be provided in front of the driver's seat 3 to recognize the face of the driver and associate the output of the sensor 10 wit′ the driver. In this case, face data of the driver may be registered in the memory 13. Further, in step S1, the control device 15 may cause the memory 13 of the operation unit 16 to store the operation by the driver.

The control device 15 determines whether to perform disinfection on the basis of whether the driver has coughed or sneezed or whether the driver is generating heat (step S2). Here, the process proceeds to step S3 on the assumption that the driver is not generating heat and has not coughed or sneezed.

The control device 15 determines whether the engine of the towing vehicle 1 is being driven (step S3). Here, step S1 is executed again, and the process proceeds to step S2 on the assumption that the engine of the towing vehicle 1 is being driven.

Here, the control device 15 determines that the driver has coughed or sneezed on the basis of the detection result of the sensor 10 and proceeds to step S4.

The control device 15 drives the irradiation device 8 and the fluid supply device 9 to neutralize the cockpit 2 (step S4). Here, in the case where the fluid supply device 9 is the gas supply device, the control device 15 irradiates the operation unit 16 with the far ultraviolet C waves by the irradiation device 8 before gas supply by the gas supply device. Thereby, it is possible to prevent soil for which disinfection has not been performed yet and to which the bacteria and viruses adhere from diffusing into the cockpit 2. Note that a suction port may be provided in the cockpit 2, and the inside of the cockpit 2 may be ventilated by a ventilator (not illustrated).

After disinfecting the soil attached to the operation unit 16 by the irradiation device 8, the control device 15 removes the soil by the gas supply device and irradiates the operation unit 16 from which the soil has been removed with far ultraviolet C waves. The far ultraviolet C waves have the wavelength of 205 to 230 nm, favorably the wavelength of around 222 nm and are emitted by the irradiation device 8 as previously described.

In step S4, the control device 15 can neutralize the bacteria and viruses floating in the cockpit 2 by driving the irradiation angle adjustment device 11 to disinfect the entire cockpit 2. Further, because there is a possibility that the bacteria and viruses adhere to the driver's hand, the control device 15 disinfects the operation unit 16 touched by the driver on the basis of the operation history stored in the memory 13.

Further, because spray of the cough or sneeze of the driver adheres to the front surface of the cockpit 2, the control device 15 performs disinfection by the irradiation device 8 on the front surface of the cockpit 2. In this case, the control device 15 sets a time for irradiating the front surface of the cockpit 2 and the operation unit 16 touched by the driver with the far ultraviolet C waves to be longer than a time for irradiating the other portions with the far ultraviolet C waves.

The irradiation time of the far ultraviolet C wave by the irradiation device 8 can be set to several seconds to several minutes according to intensity of the far ultraviolet C waves. To reliably neutralize the bacteria and viruses in the cockpit 2, it is favorable to set the irradiation time of the far ultraviolet C waves by the irradiation device 8 to be longer than a supply time of the gas of the gas supply device. Note that the supply of gas by the gas supply device may be omitted, and the gas supply device itself may be omitted.

In the case where the fluid supply device 9 is the liquid supply device that supplies the disinfectant solution, the control device 15 can disinfect the entire cockpit 2 quickly by simultaneously performing the irradiation with the far ultraviolet C waves by the irradiation device 8 and the supply of the disinfectant solution (for example, the alcohol-based solution or the quaternary ammonium salt-based solution) by the liquid supply device. Note that the irradiation time of the far ultraviolet C waves by the irradiation device 8 and the supply time of the disinfectant solution by the liquid supply device may be made the same, or the irradiation time of the far ultraviolet C waves by the irradiation device 8 may be made longer. Further, the supply of liquid by the liquid supply device may be omitted, and the liquid supply device itself may be omitted.

After performing the disinfection in step S4, the control device 15 proceeds to step S3 (step S5). The control device 15 determines whether the engine of the towing vehicle 1 is being driven (step S5). Here, the process proceeds to step S6 on the assumption that the engine of the towing vehicle 1 is stopped.

The control device 15 checks the disinfection history and the operation history of the operation unit 16 stored in the memory 13 (step S6). The control device 15 determines to perform the disinfection in a case where a predetermined time has elapsed since the previous disinfection or in a case where the driver has changed, and the control device 15 determines that the disinfection is unnecessary in other cases (step S7). The control device 15 terminates the process of the flowchart in the case of determining that the disinfection is unnecessary.

In the case of determining to perform the disinfection in step S7, the control device 15 detects whether the driver has left the towing vehicle 1. Specifically, the control device 15 determines whether the driver has left the towing vehicle 1 on the basis of at least one detection result of detection of a lock of the towing vehicle 1, failure in detection of a face by the above-described face recognition device, or failure in detection of a load of, for example, 40 Kg or more by a load sensor (not illustrated) provided in the driver's seat 3.

When detecting that the driver is not in the cockpit 2, the control device 15 performs the disinfection (step S8). In the case of performing the disinfection by the irradiation device 8, the control device 15 performs the irradiation by increasing the intensity of the irradiation device 8 as compared with the disinfection in step S4 or using ultraviolet light having a wavelength other than the wavelength of 205 to 230 nm. In a case where the irradiation device 8 has a wavelength setting function, the wavelength may be simply switched according to the wavelength setting function. Further, irradiation devices 8 having different wavelength ranges may be provided.

In the case of performing the disinfection by the liquid supply device, the control device 15 may disinfect the inside of the cockpit 2 using the disinfectant solution other than an alcohol-based disinfectant solution and a quaternary ammonium salt-based disinfectant solution. Note that the engine of the towing vehicle 1 is stopped at the time of performing step S8, but there is no problem so long as the irradiation device 8 and the fluid supply device 9 are driven using a battery (not illustrated).

The first embodiment has been described above. The above-described embodiment can be applied to anything other than the towing vehicle 1 and can be applied to, for example, a shelter or the like. Further, the towing vehicle 1 is not limited to a dump truck and can be widely applied to a bulldozer, a loader, an excavator, a narrow hole machine, or the like.

### Second Embodiment

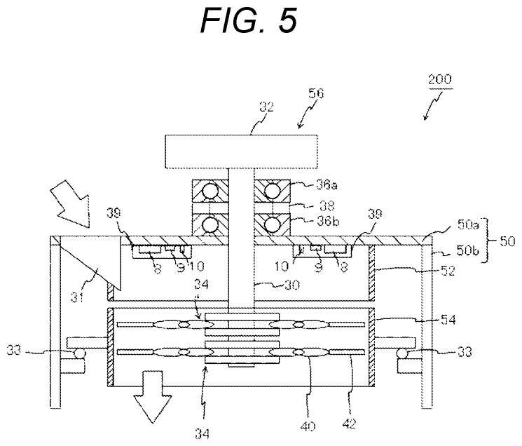

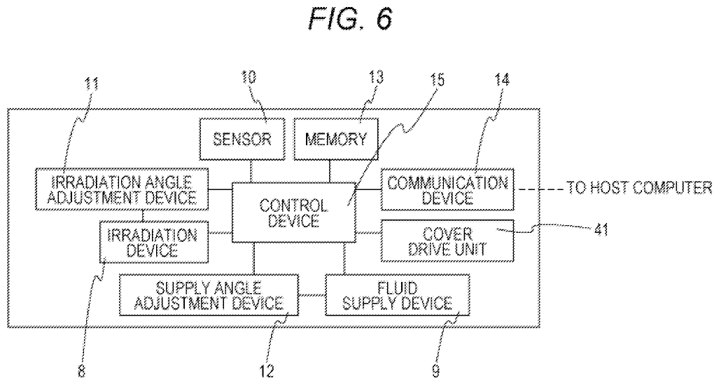

Hereinafter, a second embodiment will be described. The same components as those of the first embodiment are denoted by the same reference numerals, and the description thereof will be omitted or simplified. In the present embodiment, a disinfection device is applied to a construction machine (for example, a rotary crushing device). FIG. 5 is a schematic view illustrating a rotary crushing device 200 of the present embodiment, and FIG. 6 is a block diagram of a main part of the present embodiment. Hereinafter, the rotary crushing device 200 will be described with reference to FIGS. 5 and 6.

Rotary Crushing Device

The rotary crushing device 200 of the present embodiment is a device used for improving and effectively using raw material soil such as soil generated by construction. The rotary crushing device 200 crushes and granules the raw material soil to finely and homogeneously disperse the raw material soil. Additive materials (lime-based solidifying materials such as quicklime and slaked lime, cement-based solidifying materials such as ordinary cement and blast furnace cement, soil improving materials made of polymer materials, natural fibers, and the like) are also added into the rotary crushing device 200 as necessary. When the additive materials are added, the rotary crushing device 200 mixes the raw material soil and the additive materials to obtain improved soil, thereby adjusting properties, strength, and the like of the improved soil.

As illustrated in FIG. 5, the rotary crushing device 200 includes a frame 50, a fixed drum 52, a rotary drum 54, and a rotation mechanism 56.

The frame 50 holds each part of the rotary crushing device 200 and includes a top plate 50a and a leg 50b. The top plate 50a is, for example, an iron plate-like member, and has a function as a lid that closes an upper opening of the fixed drum 52 fixed to a lower surface (a surface on −Z side). The top plate 50a is provided with an inlet member 31 for inputting the raw material soil and additive materials (hereinafter, the raw material soil and the additive materials are referred to as processing targets) into the fixed drum 52.

The fixed drum 52 is a cylindrical container that is fixed to the lower surface (the surface on the −Z side) of the top plate 50a. The processing target is input to the fixed drum 52 via the inlet member 31, and the processing target is guided into the rotary drum 54 provided on the lower side (−Z side) of the fixed drum 52.

The rotary drum 54 is a cylindrical container that rotates (revolves) about a center axis of the cylinder (about the Z axis) by a rotary drum driving motor (not illustrated). Because the rotary drum 54 is supported by the frame 50 via a plurality of support rollers 33, the rotary drum smoothly rotates by receiving a rotational force of the rotary drum driving motor. Note that a rotation direction of the rotary drum 54 and a rotation direction of an impact member 34 may be the same rotation direction or opposite rotation directions.

A scraping rod (not illustrated) is provided inside the rotary drum 54. The scraping rod is in contact with an inner peripheral surface of the rotary drum 54 and is fixed to the fixed drum 52. Therefore, when the rotary drum 54 rotates, the scraping rod relatively moves along the inner peripheral surface of the rotary drum 54. As a result, even when the processing target adheres to the inner peripheral surface of the rotary drum 54, the processing target is scraped off by the scraping rod as the rotary drum 54 rotates. That is, the scraping rod and the rotary drum 54 that moves with respect to the scraping rod implement a function as a scraping unit that scrapes the processing target adhering to the inner peripheral surface of the rotary drum 54.

The rotation mechanism 56 includes a rotation shaft 30 disposed at the center of the fixed drum 52 and the rotary drum 54 and extending in a vertical direction (Z-axis direction), a pulley 32 provided at an upper end of the rotation shaft 30, and two-stage impact members 34 provided up and down in two stages near a lower end of the rotation shaft 30.

The rotation shaft 30 is a columnar member that is retained by the top plate 50a in a state of penetrating the top plate 50a of the frame 50 and in a state of being rotatable via two ball bearings 36a and 36b provided on the upper surface side of the top plate 50a. A spacer 38 is provided between the two ball bearings 36a and 36b, and a predetermined interval is formed between the ball bearings 36a and 36b. The lower end of the rotation shaft 30 is located inside the rotary drum 54 and is a free end. That is, the rotation shaft 30 is cantilevered.

The impact member 34 is centrifugally rotated by the rotation of the rotation shaft 30, and a thick plate 42 moves at a high speed in the vicinity of the inner peripheral surface of the rotary drum 54, thereby crushing or mixing the processing target. For this reason, the rotary crushing device 200 can also be referred to as a rotary crushing and mixing device. Note that the number of chains 40 and the thick plates 42 of the impact member 34 can be adjusted according to type and property of the raw material soil, a treatment amount, types and amounts of the additive materials, target quality of improved soil, and the like.

According to the rotary crushing device 200 of the present embodiment, the processing target input to the fixed drum 52 via the inlet member 31 is crushed and mixed by the impact member 34 in the rotary drum 54. The mixture is discharged to a lower side of the rotary drum 54.

In the present embodiment, two irradiation devices 8, two fluid supply devices 9, and two sensors 10 are provided on a lower surface side of the top plate 50a. Note that the numbers of the irradiation devices 8, the fluid supply devices 9, and the sensors 10 may be one or three or more. Further, attachment positions of the irradiation device 8, the fluid supply device 9, and the sensor 10 in X and Y directions can also be appropriately set.

In the present embodiment, the irradiation devices 8, the fluid supply devices 9, and the sensors 10 are provided not on the rotary drum 54 but on the lower surface side of the non-rotating top plate 50a. Therefore, it is possible to reduce failure of the irradiation device 8, the fluid supply device 9, and the sensor 10 due to collision of the crushed raw material soil with the irradiation device 8, the fluid supply device 9, and the sensor 10. However, there is a possibility that additives fly up to the lower surface side of the top plate 50a, or the raw material soil and the additives input to the inlet member 31 hit the irradiation device 8, the fluid supply device 9, and the sensor 10.

Therefore, in the present embodiment, a cover 39 for protecting the irradiation device 8, the fluid supply device 9, and the sensor 10 is provided. The irradiation device 8, the fluid supply device 9, and the sensor 10 are covered except when the irradiation device 8, the fluid supply device 9, and the sensor 10 are driver, The cover 39 is opened by a cover drive unit 41 when the irradiation device 8, the fluid supply device 9, and the sensor 10 are driven.

Note that the cover 39 protects the irradiation device 8, the fluid supply device 9, and the sensor 10 in FIG. 5. However, the cover 39 may be provided for each of the irradiation device 8, the fluid supply device 9, and the sensor 10. By using iron or stainless steel as the material of the cover 39, it is possible to prevent the cover 39 from being broken by the raw material soil crushed by the impact member 34.

To perform maintenance work such as cleaning of the rotary drum 54 and replacement of the chain 40 and the thick plate 42 of the impact member 34 in the rotary drum 54 when the rotary crushing device 200 is stopped, an operator may enter the rotary drum 54 from a path (not illustrated).

Therefore, in the present embodiment, when the operator enters the rotary drum 54, the sensor 10 may detect a cough or a sneeze of the operator. In the case where the cough or the sneeze is detected, the irradiation device 8 and the fluid supply device 9 are driven to disinfect the inside of the rotary drum 54. As described above, in the present embodiment, the inside of the rotary drum 54 is a disinfection area.

Description of Flowchart

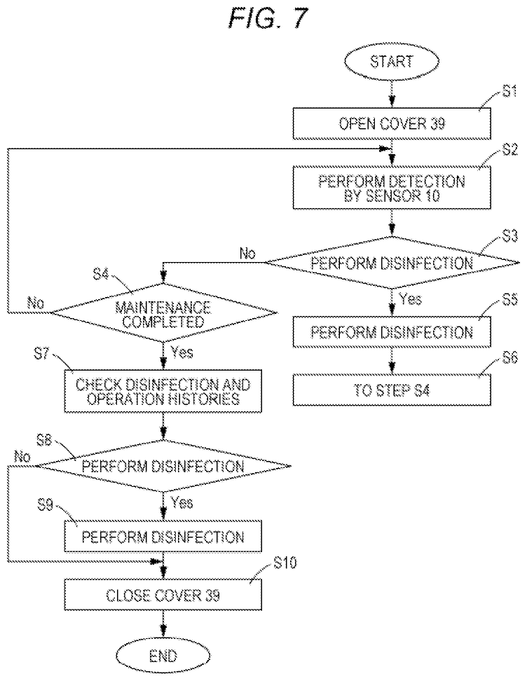

Control for neutralizing bacteria and viruses in a maintenance area by a control device 15 of the present embodiment configured as described above will be described with reference to the flowchart of FIG. 7. Note that the process of the flowchart is performed when maintenance of the rotary crushing device 200 is started.

The control device 15 drives the cover drive unit 41 to open the cover 39 (step S1). Next, the control device 15 performs detection by the sensor 10 to detect whether the operator has coughed or sneezed (step S2).

The control device 15 determines whether to perform disinfection on the basis of whether the operator has coughed or sneezed (step S3). Here, the process proceeds to step S4 on the assumption that the operator is not generating heat and has not coughed or sneezed.

The control device 15 determines whether the maintenance has been completed (step S4). Here, step S2 is executed again, and the process proceeds to step S3 on the assumption that the maintenance is continuing. Note that the start and end of the maintenance may be determined by providing a human sensor (for example, an infrared sensor) in the rotary crushing device 200 and determining whether there is an operator in the rotary drum 54.

Here, the control device 15 determines that the operator has coughed or sneezed on the basis of the detection result of the sensor 10 and proceeds to step S5 (step S3).

The control device 15 drives the irradiation device 8 and the fluid supply device 9 to neutralize the inside of the rotary drum 54 (step S5). Here, in a case where the fluid supply device 9 is a gas supply device, the control device 15 irradiates the inside of the rotary drum 54 with far ultraviolet C waves by the irradiation device 8 prior to gas supply by the gas supply device. Thereby, it is possible to prevent soil for which disinfection has not been performed yet and to which the bacteria and viruses adhere from diffusing into the rotary drum 54. Note that a suction port may be provided in the rotary drum 54, and the inside of the rotary drum 54 may be ventilated by a ventilator (not illustrated).

After disinfecting the soil attached to the impact member 34, an inner wall of the rotary drum 54, and the like by the irradiation device 8, the control device 15 removes the soil by the gas supply device and irradiates the impact member 34, the inner wall of the rotary drum 54, and the like from which the soil has been removed with the far ultraviolet C waves having a wavelength of 205 to 230 nm, favorably a wavelength of around 222 nm provided by the irradiation device 8.

In step S5, the control device 15 can neutralize the bacteria and viruses floating in the rotary drum 54 by driving the irradiation angle adjustment device 11 to disinfect the inside of the entire rotary drum 54.

Further, because spray of the cough or sneeze of the operator adheres to a side wall of the rotary drum 54 and the impact member 34, the control device 15 disinfects the side wall of the rotary drum 54 and the impact member 34 by the irradiation device 8.

In the case where the fluid supply device 9 is a liquid supply device that supplies a disinfectant solution, the control device 15 can disinfect the entire rotary drum 54 quickly by simultaneously performing irradiation with the far ultraviolet C waves by the irradiation device 8 and supply of the disinfectant solution (for example, an alcohol-based solution or a quaternary ammonium salt-based solution) by the liquid supply device.

After performing the disinfection in step S5, the control device 15 proceeds to step S4 (step S6). The control device 15 determines whether the maintenance has been completed (step S4). Here, the process proceeds to step S7 on the assumption that the maintenance has been completed.

The control device 15 checks a disinfection history and an operation history (part replacement history) stored in a memory 13 (step S7). The control device 15 determines to perform the disinfection in a case where a predetermined time has elapsed since the previous disinfection or in a case where the operator has changed, and the control device 15 determines that the disinfection is unnecessary in other cases (step S8). The control device 15 proceeds to step S10 in the case of determining that disinfection is unnecessary.

In the case of determining to perform the disinfection in step S8, the control device 15 detects whether the operator has left the rotary drum 54. The above-described human sensor may be used for this detection.

When detecting that the operator is not in the rotary drum 54, the control device 15 performs the disinfection (step S9). In the case of performing the disinfection by the irradiation device 8, the control device 15 performs irradiation by increasing intensity of the irradiation device 8 as compared with the disinfection in step S5 or using ultraviolet light having a wavelength other than the wavelength of 205 to 230 nm.

In the case of performing the disinfection by the liquid supply device, the control device 15 may disinfect the inside of the rotary drum 54 using the disinfectant solution other than the alcohol-based disinfectant solution and the quaternary ammonium salt-based disinfectant solution.

The control device 15 closes the cover 39 by the cover drive unit 41, and the control device 15 terminates the process of the flowchart.

Note that the present embodiment can be widely applied to construction machines such as a piling machine and a shielding machine.

The above-described embodiments are merely examples for describing the present invention, and various modifications can be made without departing from the scope of the present invention. Specifically, the thermometer or the face recognition device described in the first embodiment may be applied to the second embodiment. Further, the first embodiment and the second embodiment can be appropriately combined and used. Further, the control device 15 may increase the irradiation time of the irradiation device 8 or increase the amount of fluid supplied by the fluid supply device 9 with an increase in the number of times the sensor 10 detects a cough or a sneeze. Further, the control device 15 may increase the irradiation time of the irradiation device 8 or increase the amount of fluid supplied by the fluid supply device 9 with an increase in the volume of the cough or the sneeze detected by the sensor 10.

The following is a list of reference numbers used in the drawings and this description.
1 Towing vehicle
2 Cockpit
3 Driver's seat
8 Irradiation device
9 Fluid supply device
10 Sensor
15 Control device
16 Operation unit
20 Scraper vehicle
34 Impact member
50 Frame
54 Rotary drum
100 Towed scraper
200 Rotary crushing device

1-10. (canceled)

11. A construction machine comprising:
a disinfection device configured to perform at least one of supply of a disinfectant fluid or irradiation with invisible light in a maintenance area where maintenance is performed.

12. The construction machine according to claim 11, wherein the disinfection device performs at least one of the supply of the fluid or the irradiation with the invisible light from above the maintenance area.

13. The construction machine according to claim 11, wherein the invisible light is C-wave ultraviolet light having a wavelength of 205 to 230 nm.

14. The construction machine according to claim 13, wherein the disinfection device emits the C-wave ultraviolet light during the maintenance.

15. The construction machine according to claim 11, further comprising:
a rotation unit configured to perform rotation; and
a non-rotation unit configured not to perform rotation, wherein the disinfection device is provided in the non-rotation unit.

16. (canceled)

17. (canceled)

18. A disinfection device, comprising:
a detector that detects whether a person is present in a disinfection area;
an irradiation device that is capable of irradiating invisible light of a first wavelength and invisible light of a second wavelength that is different from the first wavelength; and
a controller that irradiates the disinfection area with the first wavelength by the irradiation device when the detector detects the person in the disinfection area and irradiates the disinfection area with the second wavelength when the detector does not detect the person in the disinfection area.

19. The disinfection device according to claim 18, wherein the controller determines whether irradiation with the second wavelength should be performed based on an irradiation history of the first wavelength by the irradiation device.

20. The disinfection device according to claim 18, wherein the irradiation device irradiates a wavelength of 205 to 230 nm as the first wavelength and a wavelength other than 205 to 230 nm as the second wavelength.

21. The disinfection device according to claim 18, wherein the detector comprises at least one of a microphone, an acceleration sensor, or a thermometer.

22. The disinfection device according to claim 18, further comprising:
a communication device that communicates a detection result of the detector to a host computer.

23. The disinfection device according to claim 21, further comprising:
a face recognition device that recognizes a face, wherein the controller associates a detection result of at least one of the microphone, the acceleration sensor, or a thermometer to a detection result of the face recognition device.

24. The disinfection device according to claim 18, further comprising:
a face recognition device that recognizes a face, wherein the controller determines whether irradiation by the second wavelength should be performed based on a detection result of the face recognition device.

25. The disinfection device according to claim 18, wherein the detector comprises a load sensor provided in a seat.

26. The disinfection device according to claim 18, wherein:
the disinfection area is provided an operation unit that is operated by the person, and
the controller increases irradiation time of the operation unit by the irradiation device based on the detection result of the detector, longer than the irradiation time of other parts of the operation unit.

27. The disinfection device according to claim 18, wherein the irradiation device makes intensity of irradiation by the second wavelength stronger than intensity of irradiation by the first wavelength.
